(19) United States
(12) Patent Application Publication
(10) Pub. No.: US 2005/0074477 A1
(43) Pub. Date: Apr. 7, 2005

(54) Method for the treatment of diseased, degenerated, or damaged tissue using three dimensional tissue produced in vitro in combination with tissue cells and/or exogenic factors

(76) Inventors: Olivera Josimovic-Alasevic, Berlin (DE); Jeannette Libera, Berlin (DE); Hans-Peter Wiesmann, Ochtrup (DE); Ulrich Joos, Munster (DE); Gordana Vunjak-Novakovic, Belmont, MA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/501,520

(22) PCT Filed: Nov. 7, 2003
(86) PCT No.: PCT/DE03/03765

(30) Foreign Application Priority Data
Nov. 7, 2002 (DE) 10253066.1

(51) Int. Cl. C12N 5/08; A61F 2/00

(52) U.S. Cl. 424/423; 435/366

(57) ABSTRACT

The invention relates to a tissue replacement structure comprising (a) a preformed three-dimensional tissue which can be produced by obtaining cells from a human or animal organism and culturing them in a stationary fashion as a suspension culture in cell culture vessels with hydrophobic surface and tapering bottom until a cell aggregate is formed which has differentiated cells embedded therein and has an outer region wherein cells capable of proliferation and migration are present; (b) (i) an autologous cell suspension which can be produced from endogenous cells, with endogenous serum being added, with no addition of growth-promoting compounds, (ii) implants or support materials and/or (iii) growth factors; and/or (c) can be obtained by exposure of the tissue according to (a) to electromagnetic fields, mechanical stimulation and/or ultrasound.

The invention relates to a new tissue replacement structure, to a method of modifying a tissue lesion, and to the use of preformed three-dimensional tissue as a source of messenger substances and/or structural components.

Hyaline cartilage tissue consists of one single type of cells, i.e., chondrocytes which synthesize an elastic extracellular matrix (ECM). Healthy ECM is mainly composed of collagens and proteoglycans (PG). The collagen prevailing in hyaline cartilage is type II collagen which forms highly elastic fibers. Proteoglycans provide for crosslinking of the collagen fibers. In healthy cartilage, there is a continuous conversion of matrix components which is important for constant elasticity of the cartilage.

One important function for ECM metabolism is that of enzymes and inhibitors thereof. Enzymes effective in cartilage are metalloproteinases (MMPs) which catalyze the degradation of collagens and proteoglycans. The activity of these enzymes is regulated via inhibitors (tissue inhibitors of metalloproteinases: TIMPs) likewise synthesized in cartilage. Equilibrium between MMPs and TIMPs is crucial for maintaining the cartilage matrix.

Cytokines and growth factors have an influence on the synthesis of cartilage matrix structural components and of degrading enzymes and inhibitors thereof. In healthy cartilage, there is an equilibrium between degradation and de novo synthesis of matrix components and thus between the expression of cytokines and growth factors, which equilibrium is crucial for maintaining cartilage elasticity, ensuring continuous renewal of “consumed” structural components. Augmented presence of growth factors in a joint may support the in vivo regenerative capability of cartilage.

The most important anabolic growth factors known in cartilage are transforming growth factor β (TGFβ), platelet-derived growth factor (PDGF), fibroblast growth factor 2 (FGF2; formerly basic (b) FGF), insulin-like growth factor (IGF), and bone morphogenetic proteins (BMPs). TGFP, IGF I and BMP-2 are considered the most important factors for promoting cartilage maturing.

Both PDGF and IGF stimulate the growth of human chondrocytes. IGF I is the dominant growth factor in adult tissue, promoting PG synthesis and inhibiting degradation of cartilage matrix even upon stimulation with cartilage-degrading cytokine IL-1β.

TGFβ₁ has an anabolic effect in the cartilage metabolism, stimulating the expression of TIMP, the PG and collagen synthesis, and promoting the growth of chondrocytes. In addition, TGFβ₁ enhances the cartilage-regenerating effect of PDGF and IGF.

FGF 2 stimulates the proliferation of cultured chondrocytes and has a synergistic effect in combination with TGFP; stimulation of the matrix synthesis by FGF can also be detected.

BMPs stimulate the proteoglycan synthesis in chondrocytes and support the differentiation of precursor cells (e.g. from the periosteum or bone marrow) into mature chondrocytes. On the whole, they advance the differentiation of chondrocytes, thereby supporting cartilage healing.

The mechanism of action of the classical ACT technique developed by Brittberg and Peterson is based on the ability of autologous chondrocytes grown in monolayers to form a hyaline or hyaline-like regenerate in vivo, which is similar to the surrounding hyaline joint cartilage, thus representing a functional regeneration of cartilage lesions.

For the treatment of patients it is necessary to grow a small number of chondrocytes, obtained from a small biopsy, in a monolayer culture. During this process, the chondrocytes assume the typical shape of mesenchymal cells, changing their expression pattern compared to the in situ situation. Indeed, the ability of chondrocytes to re-express the markers of hyaline cartilage after growth in monolayer and subsequent transfer in 3D culture has already been established in vitro in numerous studies. Using a specifically developed cell culture system, it has also been demonstrated that chondrocytes grown in monolayer in a purely autologous fashion—without addition of periosteum or growth factors—re-express collagen II and S-100 as cartilage markers after transfer in 3D culture without carrier. In various cell culture systems, injection of growth factors promotes and enhances the synthesis of specific cartilage markers and speeds up healing of cartilage defects in animal models. It is therefore reasonable to assume that the same mechanisms will take effect in vivo after an ACT has been performed. Following application in the three-dimensional space in the joint, created by the periosteum or collagen material, the chondrocytes exhibit their former in vivo expression pattern, regenerating hyaline cartilage with marked expression of type II collagen. This was confirmed by means of biopsies taken from patients after an ACT had been performed. As already demonstrated in vitro, growth factors such as TGFβ, IGF I and BMP-2 are secreted by cultured periosteum, thus promoting the regeneration of hyaline cartilage by chondrocytes injected in the course of the ACT.

Further in vitro experiments on joint cartilage from various species have demonstrated that chondrocytes applied onto the cartilage surface in a cell suspension stably associate with the native tissue, resulting in stable and long-term integration in the surrounding native cartilage of the new cartilage formed following ACT.

In normal use of the joints, the hyaline joint cartilage coating same is exposed to enormous pressure load, and damage of its structure or injuries will have great effects on the entire functionality of the system.

The natural regenerative capacity of joint cartilage is very low. In healthy adult cartilage, the chondrocytes normally no longer divide (Mankin 64). Only joint cartilage defects where the subchondral osseous plate has been damaged have some repair capacity as a result of stem cells infusing from the medullary space. In contrast, superficial chondral defects with intact subchondral osseous plate virtually have no capacity of self-regeneration.

Once the cartilage has been damaged, the degeneration continuously expands due to stimulation of cartilage-degrading influences. Cartilage injury therefore implies an increased risk of arthrosis for an affected patient, ultimately necessitating the use of a joint endoprosthesis in many cases.

According to the statements above, solutions to restore the function of tissues or build up tissues which are or have been damaged, degenerated or affected have been sought for quite some time in the field of regenerative medicine. On the one hand, endogenous cells with and without support material, and, on the other hand, support materials exclusively have been used to this end; depending on the indication, absorbable or non-absorbable materials can be used.

The object of the invention was therefore to provide a tissue replacement structure or an in vitro tissue, particularly a cartilage replacement or cartilage regeneration structure, and a method for the treatment or modification of affected, damaged and degenerate tissue, which method would allow easy, safe, efficient and effective treatment of tissue defects, e.g. of affected, damaged and degenerate cartilage tissue.

The invention solves the above technical problem by providing a tissue replacement structure comprising
(a) a preformed three-dimensional tissue which can be produced by obtaining cells from a human or animal organism and culturing them in a stationary fashion as a suspension culture in cell culture vessels with hydrophobic surface and tapering bottom until a cell aggregate is formed which has differentiated cells embedded therein and has an outer region wherein cells capable of proliferation and migration are present;
(b) (i) an autologous cell suspension which can be produced from endogenous cells, with endogenous serum being added, with no addition of growth-promoting compounds, (ii) implants or support materials and/or (iii) growth factors; and/or
(c) can be obtained by exposure of (a) to electromagnetic fields, mechanical stimulation and/or ultrasound.

Accordingly, the invention relates to a three-dimensional tissue of varying size, i.e., in vitro tissue, used to make tissue therapy more effective, which tissues may also be referred to as spheroids. Essentially, said tissue replacement or tissue regeneration structures, or spheroids, are composed of cells contained in the spheroid and of a matrix formed by these cells and are present in combinations with single suspension cells, with genetically modified single suspension cells, with support materials, with exogenic growth factors, active substances, exogenic DNA, RNA, and/or with implants. Such spheroids can be employed as in vitro test systems for biological and chemical active substances and physical factors when treating affected, degenerate and/or damaged tissue, and as organ replacement, or as tissue replacement structures. The tissue replacement structures of the invention are used to induce and speed up tissue regeneration or to make tissue regeneration possible in the first place, e.g. in those cases where spheroids are used in combination with specific active substances, for instance in building up cardiac muscle following myocardial infarction.

While the prior art uses endogenous cells, with and without support material, or exclusively uses absorbable or non-absorbable support materials, the structures according to the invention imply the use and transplantation of in vitro produced, structurally and functionally prefabricated, three-dimensional tissues to establish organ and tissue functions, i.e., single cells according to well-known methods and structures will not be employed. The tissue replacement structures or spheroids according to the invention therefore allow transplantation of prefabricated tissue and a further increase in effectiveness by combining most various tissue spheroids with single cells and exogenic factors. Thus, unlike in the prior art, e.g. growth factors are no longer liberated by supports or support materials—regardless whether in combination with cells or without same. Surprisingly, it has been demonstrated that the new tissue replacement structures or spheroids can be used for combining with other factors promoting tissue regeneration. Particularly when using cartilage spheroids and cartilage cells according to the invention, improved genesis was achieved. Such surprisingly improved genesis was also observed when combining other spheroids and growth-promoting factors or cells.

In many diseases, tissue replacement structures or spheroids cannot be inserted in the affected tissue region in an isolated fashion because, due to the circumstances following transplantation, they do not remain in a particular location and consequently are incapable of inducing a well-directed tissue regeneration. Advantageously, the spheroids can be fixed in the respective locations. This is done with advantage by combination with a support or a membrane which itself is bound or immobilized in the defective area or in the surroundings thereof. Artificial three-dimensional tissue structures, such as the so-called cell spheres from bone cells, do not have sufficiently high mechanical strength to allow sole insertion thereof in a bone defect. The tissue replacement structures or spheroids according to the invention are introduced in combination with a three-dimensional support. Surprisingly, it has been demonstrated that spheroids give especially good interaction, adherence and integration with the support material. Advantageously, this allows good fixation of the spheroids in the defective area. Surprisingly, adhesion of the spheroids is promoted by the presence of single cells, the singles cells forming a contact bridge between the native tissue to be treated and the spheroids or tissue replacement structures. In particular, this has been demonstrated in the use of cartilage aggregates with cartilage cells on and in native cartilage tissue. According to the invention, the single cells or endogenous cells can be modified by genetic engineering in order to promote the tissue regeneration process, for example. Especially in those cases where spheroids defy transfection by genetic engineering, the effect of promoting tissue regeneration can be achieved by administering genetically engineered cells in the defective area.

Preferably, the regeneration process effected by using the tissue replacement structures of the invention can also be employed subsequent to transplantation of the spheroid into the tissue to be treated, using a combination of spheroid and growth factors or other factors if, for example, modifications by genetic engineering are undesirable. For example, DNA or RNA molecules can be used as factors which, e.g. following non-specific incorporation by the cells, can also give rise to synthesis of the corresponding sequences.

Another advantage of the structures according to the invention is that they can also be used as a test system for medicaments. In particular, this also applies to those cases where the spheroids are obtained from affected cells, e.g. from arthritic cartilage cells, or from tumor cells, or from muscle cells in cases of muscular dystrophy, which cells are used to investigate active substances and medicaments. In addition to their rapid effect and their use both in vivo and in vitro, another advantage of the tissue replacement structures according to the invention is represented by the fact that patients, which can be humans or animals, can be treated in a purely autologous fashion, thus excluding the risk of defence reactions to an incorporated graft. In particular, hospital and rehabilitation periods are significantly reduced in this way. Also, the cost of the overall regeneration process is reduced, and more rapid rehabilitation of treated patients is achieved. Furthermore, the structures according to the invention can be used in screening of active substances or generally as an in vivo or in vitro test system, e.g. in testing drugs for their influence on tissue regeneration.

Preferably, the following can be used as cells in such tissue: muscle cells (striated cardiac muscle, skeleton muscle and smooth muscle cells), cartilage cells (from hyaline cartilage, fibrous cartilage, elastic cartilage), bone cells (osteoblasts and osteocytes), skin cells (keratinocytes, e.g. spinous cells), connective tissue cells from corium and subcutis, cells from eccrine and apocrine sudoriferous glands and sebaceous glands, cells from the hair rudiment (e.g. mitotically active hair bulb cells, cells from the nail rudiment), endothelial cells, connective tissue cells (fibroblasts, fibrocytes, wandering cells, mast cells, pigment cells, reticular cells), fat cells (adult fat cells and fat precursor cells), nervous tissue cells (nerve cells, neuroglia cells), mesenchymal stem cells from bone marrow/peripheral blood, liver cells, epithelial cells from monolayer and multilayer epithelia and surface epithelia, gangetic epithelia, glandular epithelia, sensory epithelia, endoepithelia (cells from the stratum superficiale, stratum intermedium, stratum basale, stratum corneum, stratum granulosum, stratum spinosum) and/or pancreatic cells.

Preferably, the following can be used as cells to be combined with tissue: muscle cells (striated cardiac muscle, skeleton muscle and smooth muscle cells), cartilage cells (from hyaline cartilage, fibrous cartilage, elastic cartilage), bone cells (osteoblasts and osteocytes), skin cells (e.g. keratinocytes), endothelial cells, connective tissue cells (tendons and ligaments), fat cells (adult fat cells and fat precursor cells), nervous tissue cells (nerve cells, neuroglia cells), stem cells (from bone marrow/peri-pheral blood, from adult tissues per se, e.g. pancreas, cornea, from embryos and fetes), liver cells, epithelial cells from monolayer and multilayer epithelia and surface epithelia, gangetic epithelia, glandular epithelia, sensory epithelia, endoepithelia (cells from the stratum superficiale, stratum intermedium, stratum basale, stratum corneum, stratum granulosum, stratum spinosum) and/or pancreatic cells. The cells in the tissue, i.e., the preformed three-dimensional tissue, and the single cells from the tissue cell suspension can be modified by genetic engineering. The genetic modification can be such that growth factors, cytokines, structural proteins, marker proteins, or regulatory active substances are expressed, in particular.

Advantageously, the structures according to the invention can be combined with implants or support materials, for example:
biocompatible, degradable or non-degradable (absorbable), allogenic, autologous, xenogeneic and synthetic materials which may bear exogenic factors (such as growth factors) themselves;
polymers (for example, polylactides, polyglycolides, hyaluronic acids and all derivatives thereof,
preferably a neutral PGA/PLA mixture,
calcium carbonates, hydroxyapatites, calcium phosphates, animal pretreated natural bone matrix,
fiber proteins, fibrin-based supports,
gels (such as alginates, agarose, collagen gel, hydrogels, fibrin),
membranes, fleeces, scaffolds (3D supports), and/or
prostheses (titanium, miscellaneous metal and noble metal materials).

Furthermore, it is possible to combine the structures according to the invention and also, the tissue cell suspension or the preformed three-dimensional tissue with exogenic growth factors, where the respective tissue-specific growth factors can be used which effect the processes of tissue build-up and rearrangement at each particular site, governing or regulating same. In the case of cartilage, for example, this is one of the following factors: transforming growth factor β (TGFP), platelet-derived growth factor (PDGF), fibroblast growth factor 2 (FGF2; formerly basic (b) FGF), insulin-like growth factor (IGF), and bone morphogenetic proteins (BMPs); e.g. BMP7 in the case of bones, or MGF in the case of muscles.

In addition to exogenic growth factors, it is obviously possible to use other exogenous factors, e.g. all the substances having a regulatory effect, such as cytokines or enzymes, and also, RNA and DNA molecules, or viruses, or proteins usually produced or secreted by body cells, such as cytokines (IL-1, TNF-alpha), adhesion proteins, enzymes (lipases, proteinases), messenger substances (cAMP), matrix structural proteins (collagens, proteoglycans), proteins in general, lipids (phosphatidylserine).

In a preferred embodiment, the invention also provides a cartilage replacement structure, comprising
(a) a preformed three-dimensional cartilage tissue which can be produced by obtaining cartilage cells, bone cells, or mesenchymal stem cells from a human or animal organism and culturing them in a stationary fashion as a suspension culture in cell culture vessels with hydrophobic surface and tapering bottom until a cell aggregate is formed which includes at least 40% by volume of extracellular matrix having differentiated cells embedded therein, and which cell aggregate has an outer region wherein cells capable of proliferation and migration are present; and
(b) an autologous cartilage cell suspension produced from endogenous cells, with addition of endogenous serum and without using growth-promoting compounds, and/or exposing the tissue according to (a) to physical factors.

According to the invention, patient-derived tissue biopsies or samples, or mesenchymal stem cells, e.g. from peripheral blood or bone marrow, are used as starting material for the preformed tissue, i.e., for a component of the tissue replacement structure. The tissue-building cells are isolated from the biopsies according to conventional methods, using enzymatic digestion of the tissue, migration, or reagents recognizing the target cells. According to the invention, these cells are then subjected to stationary culturing in suspension in a simple fashion, using conventional culture medium in cell culture vessels with hydrophobic surface and tapering bottom, until a three-dimensional cell aggregate is formed which includes at least 40% by volume, preferably at least 60% by volume, and up to a maximum of 95% by volume of extracellular matrix (ECM) having differentiated cells embedded therein. The cell aggregate having formed has an outer region wherein cells capable of proliferation and migration are present.

It is noteworthy that all cells integrated in the spheroids produced according to the invention survive, and that the cells inside do not necrotize even after an advanced period of culturing. With increasing time of cultivation, the cells inside the aggregates undergo differentiation to form spheroids consisting of ECM, differentiated cells and a peripheral proliferation zone. The process of formation of the tissue-specific matrix with embedded cells is highly similar to the process of tissue formation or neogenesis and reorganization in the body. During differentiation in cell culture, the spacing between the aggregated cells increases due to formation of the tissue-specific matrix. A tissue histology develops inside the spheroids which is highly similar to natural tissue. As in natural cartilage, the cells inside the spheroids are supplied with nutrients by way of diffusion only. During the further course of spheroid production, a zone of cells capable of proliferation and migration is formed at the boundary of the spheroids. This zone is invaluably advantageous in that, following incorporation of the spheroids in defects, the cells situated in this peripheral zone are capable of migrating to make active contact with the surrounding tissue and/or enable integration of the tissue produced in vitro in the environment thereof. Thus, the tissue-specific cell aggregates produced are excellently suited for use in the treatment of tissue defects and in the in vitro and in vivo neogenesis of tissue.

Depending on the size of the tissue defect to be treated, it can be advantageous to transplant larger pieces of tissue at an early stage so as to achieve more rapid repletion of the defect. In this event, at least two, or preferably more of the cell aggregates obtained are fused by continuing culturing thereof under the same conditions and in the same culture vessels as described above until the desired size is reached.

The cartilage or bone tissue obtained is extraordinarily stable. The cell aggregates can be compressed to ¾ of their diameter without breaking or decomposing e.g. when injected into the body by means of a needle. The pieces of tissue can be taken out of the cell culture vessel using pincers or a pipette.

In an advantageous embodiment of the invention, the cells obtained from the patient are first grown in a monolayer culture in a per se known fashion to have sufficient cartilage or bone cells available for suspension culturing according to the invention. Passaging of the cells in monolayer culture is kept as low as possible. After reaching the confluent stage, the cells grown in monolayer are harvested and cultured in suspension according to the inventive method as described above.

A medium usual both for suspension and monolayer culture, e.g. Dulbecco's MEM, with addition of serum, can be used as cell culture medium. It is preferred to use DMEM and HAMS at a ratio of 1:1. However, to avoid an immunological response of the patient to the tissue produced in vitro, it is preferred to use autogenous serum from the patient as serum. It is also possible to use xenogeneic or allogenic serum.

According to the invention, no antibiotic, fungistatic agents or other auxiliary substances are added to the culture medium. It has been found that only autogenous, xenogeneic or allogenic cultivation of the cells and cell aggregates and cultivation with no antibiotic and fungistatic agents allows for non-affected morphology and differentiation of the cells in the monolayer culture and undisturbed formation of the specific matrix within the cell aggregates. Furthermore, by avoiding any additive during the production, any immunological reaction is excluded when incorporating the tissue produced in vitro in a human or animal organism.

Quite surprisingly, indeed, growth factors or other growth-stimulating additives are required neither in suspension culturing, nor in monolayer culturing. Despite the absence of such additives, three-dimensional cell aggregates with tissue-specific properties are obtained after only two days of suspension culturing according to the invention. Obviously, the size depends on the number of introduced cells per volume of culture medium. For example, when incorporating 1×10⁷ cells in 300 μl of culture medium, three-dimensional spheroids about 500-700 μm in diameter are formed within one week. For a tissue defect of 1 cm², it would be necessary to transplant about 100 of such spheroids, e.g. by injection. Another way would be in vitro fusion of small cell aggregates to form larger ones—as described above—and incorporation of the latter in the defect. According to the invention, it is preferred to use between 1×10⁴ and 1×10⁷ cells in 300 μl of culture medium to produce the small cell aggregates, more preferably 1×10⁵ cells. Depending on the cell type and patient-specific characteristics, the spheroids having formed after several days are then cultured in a suitable culture medium for at least 2-4 weeks to induce formation of the tissue-specific matrix. From about one week of culturing on, it is possible to fuse individual spheroids in special cases, so as to increase the size of the tissue patch.

As cell culture vessels, the inventive cultivation in suspension requires the use of those having a hydrophobic, i.e., adhesion-preventing surface, such as polystyrene or Teflon. Cell culture vessels with a non-hydrophobic surface can be hydrophobized by coating with agar or agarose. Further additives are not required. Preferably, well plates are used as cell culture vessels. For example, 96-well plates can be used to produce small cell aggregates, and 24-well plates to produce said fused aggregates.

According to the invention, the cell culture vessels must have a tapering, preferably concave bottom. It has been found that the tissue of the invention will not be formed in flat-bottomed vessels. Apparently, the depression is useful in finding the cells. In combination with the tissue cell suspension, preferably the cartilage cell suspension, the preformed three-dimensional tissue thus obtained is forming the tissue replacement structure, preferably cartilage replacement structure. However, it is also preferred to use the preformed three-dimensional tissue in combination with support materials or growth factors. Furthermore, the preformed tissue is preferably exposed to physical forces such as electromagnetic fields, mechanical stimulation and/or ultrasound. These physical forces can act on the preformed tissue during the production of the replacement structure in vitro—e.g. in the culture vessel —or in vivo, i.e., in the patient.

In a preferred fashion, the tissue replacement structure is a muscle replacement structure, particularly a cardiac smooth muscle replacement structure, or a bone replacement structure.

The invention also relates to a method of modifying a tissue lesion, in which method
(a) an autologous cell suspension produced from endogenous cells, with addition of endogenous serum and without adding growth-promoting compounds, and
(b) a preformed three-dimensional tissue which can be produced by obtaining cells from a human or animal organism and culturing them in a stationary fashion as a suspension culture in cell culture vessels with hydrophobic surface and tapering bottom until a cell aggregate is formed which has differentiated cells embedded therein and has an outer region wherein cells capable of proliferation and migration are present;
are incorporated in the tissue lesion and/or
(c) exposure of the tissue according to (a) to electromagnetic fields, mechanical stimulation and/or ultrasound is effected in vivo or in vitro.

In another preferred embodiment the invention relates to a method of modifying a cartilage lesion, in which method
(a) an autologous cartilage suspension produced from endogenous cells, with addition of endogenous serum and without adding growth-promoting compounds, and
(b) a preformed three-dimensional cartilage tissue which can be produced by obtaining cartilage cells, bone cells, or mesenchymal stem cells from a human or animal organism and culturing them in a stationary fashion as a suspension culture in cell culture vessels with hydrophobic surface and tapering bottom until a cell aggregate is formed which includes at least 40% by volume of extracellular matrix, which cell aggregate has differentiated cells embedded therein and has an outer region wherein cells capable of proliferation and migration are present;
   are incorporated in the cartilage lesion and/or exposure of the tissue according to (a) to physical factors is effected in vitro or in vivo.

Preferably, the tissue lesion is a bone, cartilage and/or muscle lesion.

The method of the invention utilizes the natural effect of growth factors supporting cartilage regeneration, in order to speed up the treatment of the defect, particularly in comparison to the classical therapy. Using said three-dimensional tissue, especially cartilage tissue, it is possible to achieve expression of completely natural autologous growth factors directly in the treated defect, thus speeding up the formation of functional regenerate.

Accordingly, in the course of a treatment for the modification of a tissue lesion, especially a cartilage lesion, a preformed three-dimensional cartilage tissue is applied in addition to an autologous cartilage cell suspension, said three-dimensional cartilage tissue synthesizing the growth factors required for the stimulation of matrix synthesis, thereby supporting healing or modification of the treated tissue lesion, e.g. a cartilage lesion. The cells of the cartilage suspension incorporated together with the three-dimensional cartilage tissue—which may also be referred to as 3D construct—ensure optimum integration of the regenerate being formed, particularly in the surrounding cartilage. The growth factors synthesized by the three-dimensional tissue give rise to an increased stimulation of matrix formation of the suspension cells, for example, thus speeding up healing of the defect.

The method according to the invention is particularly advantageous because a three-dimensional cartilage tissue is preformed even in vitro, under completely autologous conditions, without addition of substances not being derived from the patient himself, which tissue is highly similar in its properties to native cartilage, thereby providing the basis for further build-up of cartilage substance immediately after operation.

Another advantage is that the complex application of the periosteal flap according to familiar methods can thus be avoided, because the growth factors secreted by the periosteum—essential to the mechanism of action in the well-known methods—are provided by the preformed three-dimensional cartilage tissue in the method of the invention. According to the invention, it has been demonstrated that the preformed three-dimensional cartilage tissue is capable of forming a hyaline cartilage matrix even in vitro. Collagen II, in particular, being the characteristic protein of hyaline joint cartilage, is formed in large quantities by the preformed three-dimensional cartilage tissue, and above all, the growth factors are already produced in an active fashion at the time of transplantation.

In a special embodiment of the invention, incorporation of the cartilage cell suspension and cartilage tissue is followed by covering the lesion with a membrane.

The invention also relates to the use of cartilage cells, muscle cells, bone cells, or mesenchymal stem cells obtained from a human or animal organism and cultured in a stationary fashion as a suspension culture in cell culture vessels with hydrophobic surface and tapering bottom until a cell aggregate is formed which, in particular, includes at least 40% by volume of extracellular matrix, has differentiated cells embedded therein, and has an outer region wherein cells capable of proliferation and migration are present, as a source of messenger substances, structural, scaffold and/or matrix components, especially growth factors and/or cytokines.

By using the resulting cartilage cells as a source of regeneration-promoting growth factors and already preformed hyaline cartilage matrix, it is possible to achieve significantly more rapid healing of cartilage defects than is possible with methods known to date. In addition to the rapid effect, one essential advantage offered by the in vivo or in vitro use is represented by the fact that patients can be treated in a purely autologous fashion, thus excluding the risk of defence reactions to the incorporated graft.

In another preferred embodiment of the invention, the use is in vivo or in vitro.

In another, particularly preferred embodiment the use is in the treatment of a tissue lesion, preferably a cartilage, bone and/or muscle lesion.

In the meaning of the invention, a lesion is understood to include any disease, degeneration or damage of cells or tissue structures. Thus, the structures of the invention can preferably be used in the treatment of the following diseases, degenerations or damages:
cardiac muscle lesions,
arthrosis (for example, apply spheroids on cartilage surface and cover with a membrane),
rheumatism, arthritis,
diseases based on genetic defects or changes,
infarctions (intravital tissue necroses, e.g. spleen infarction),
ischemias (e.g. due to arterial occlusion),
malformations, lesions and degeneration of organs/tissues of the nervous system and neuromuscular system,
diseases and degeneration of tissues in the eye (e.g. cornea, conjunctiva), e.g. retinal detachment,
diseases and degeneration of the neuroendocrine system (e.g. hypothyreoses of the thyroid gland),
cardiovascular system (e.g. malformations on the heart, cardiac infarction),
lesions of the respiratory tract,
digestive tract (esophagitis, e.g. formation of gastric mucosa following gastritides),
bones: non-healing fractures, bone formation following tumors,
joints: meniscus diseases and lesions, intervertebral disks, tendons, ligaments, and
skin lesions (e.g. hypotrichoses).

From the disclosure of the use according to the invention, other equivalent uses will be apparent to those skilled in the art. The tissue replacement structure according to the invention, i.e., the combination preparation comprising the preformed three-dimensional tissue and the respective additive, i.e., the tissue cell suspension, implant or support material or growth factor, can be used for any tissue from which cells can be isolated and used separately or in the production of said preformed three-dimensional tissue. Of course, physical forces such as electromagnetic fields, mechanical stimulation and/or ultrasound can also be used as an additive for the preformed three-dimensional tissue in the meaning of the invention. In this event, the preformed three-dimensional tissue is exposed in vitro or in vivo to said physical forces in such a way that healing of the lesion or defect takes place.

Furthermore, the tissue replacement structures of the invention can also be used as organ replacement, e.g. in restoring one or more organ functions of the above-mentioned tissues. Other preferred organs or tissues are dopamine-producing structures and tissues in the treatment of Parkinson's disease or nerve degeneration diseases, insulin-producing structures in the treatment of pancreas defects, thyroxine-producing tissues in the treatment of thyroid defects, and also, liberin- or statin-producing replacement structures in restoring the hypothalamus function.

The invention also relates to a tissue replacement structure selected from the group of muscle, connective, skin, fat, nervous, liver tissues, endothelia, epithelia, and/or stem cells, which structure can be produced by obtaining cells from a human or animal organism and culturing them in a stationary fashion as a suspension culture in cell culture vessels with hydrophobic surface and tapering bottom until a cell aggregate is formed which has differentiated cells embedded therein and has an outer region wherein cells capable of proliferation and migration are present.

The invention also relates to a kit comprising the structures of the invention, and to the use thereof in diagnosis and therapy. In addition, the kit may include buffers, serums, salts, culture media, as well as information how to combine the contents.

Thus, the invention relates to a tissue replacement structure and to a method for the modification or treatment of tissue lesions, e.g. cartilage lesions, using exclusively endogenous three-dimensional cultured cartilage in the form of so-called spheroids; for example, restoration of degenerate arthritic cartilage is possible in this way. Using this spheroid technology or the spheroids, a platform technology for further extensive product innovation is provided, allowing endogenous cell regeneration of traumatic joint cartilage lesions. The use of endogenous growth factors produced by spheroids results in substantially more rapid formation of pressure-resistant joint cartilage. In particular, this is achieved by well-directed mono-specific growth of cartilage, thereby allowing minimal invasive, arthroscopic autologous chondrocyte transplantation treatment. More particularly, the hospital and rehabilitation periods are significantly reduced. Also, costs are reduced, and more rapid rehabilitation of treated patients is achieved. Obviously, the spheroid technology is not restricted to cartilage, but rather can be used for the regeneration of any type of human tissue.

Without intending to be limiting, the invention will be illustrated in more detail with reference to the examples.

### EXAMPLES

Preparation of a First Component (Cartilage) of the Combination Preparation (Tissue Replacement Structure)

A biopsy is taken from a patient from a region of hyaline, healthy cartilage. Chondrocytes are isolated from this biopsy, using enzymatic digestion by incubation with collagenase solution. Following separation of the isolated cells from undigested cartilage tissue, the cells are transferred in cell culture flasks and, following addition of DMEM/HAMS F12 culture medium (1/1) and 10% autologous serum from the patient, incubated at 37° C. and 5% CO₂. The medium is exchanged twice a week. After reaching the confluent stage, the cell layer is washed with physiological saline solution and harvested from the cell culture surface using trypsin. Following another washing, 1×10⁵ cells each time are transferred in a cell culture vessel coated with agarose. After one day, the first cells arrange into aggregates. These aggregates are supplied with fresh medium every second day and cultured for at least 2 weeks.

After only one week, type II collagen and proteoglycans were detected in the aggregates. To this end, a specific antibody to type II collagen was used. The primary antibody bound to type II collagen was detected using a second antibody and an ABC system coupled thereto. That is, the second antibody has coupled the enzyme alkaline phosphatase via avidin-biotin thereto, which enzyme effects reaction of the substrate fuchsin to form a red dye.

The proteoglycans were detected by means of Goldner staining. Type II collagen and proteoglycans are components of the cartilage matrix in vivo, representing the most important structural proteins which are of crucial significance for cartilage function.

At the same time, the protein S 100 specific for cartilage cells was detected in the outer layer of the aggregates. S 100 is neither expressed in bone tissue nor in connective tissue. It is only these latter tissues which also could have formed. Consequently, the tissue having developed was unambiguously proven to be cartilage tissue.

After culturing for 1-2 weeks, the cells are still close together. With increasing cultivation time, the proportion of extracellular matrix increases and the proportion of cells decreases. After one week, at least 40% ECM can be detected, and after 3 weeks, about 60% ECM has already developed. After 3 months of cartilage tissue cultivation, the proportion of ECM has increased to 80-90%. That is, cartilage-like tissue has been built up inside the aggregates produced, which tissue in its structure corresponds to in vivo cartilage and is also capable of assuming the function of cartilage tissue.

Preparation of another First Component (Bone Tissue)

A bone biopsy is taken from a patient from a spongiosa region. Osteoblasts are isolated from this biopsy, using enzymatic digestion by incubation with collagenase solution. Following separation of the isolated cells from the undigested bone tissue, the cells are transferred in cell culture flasks and, following addition of DMEM/HAMS F12 culture medium (1/1) and 10% autologous serum from the patient, incubated at 37° C. and 5% CO₂. The medium is exchanged twice a week. After reaching the confluent stage, the cell layer is washed with physiological saline solution and harvested from the cell culture surface using trypsin. Following another washing, 1×10⁵ cells each time are transferred in a cell culture vessel coated with agarose. After one day, the first cells arrange into aggregates. These aggregates are supplied with fresh medium every second day and cultured for at least 2 weeks.

After only one week, type I collagen and proteoglycans were detected in the aggregates. To this end, a specific antibody to type I collagen was used. By detecting collagen I, unambiguous proof is provided that this is not cartilage tissue. The primary antibody bound to type I collagen was detected using a second antibody and an ABC system coupled thereto. That is, the second antibody has coupled the enzyme alkaline phosphatase via avidin-biotin thereto, which enzyme effects reaction of the substrate fuchsin to form a red dye.

As in Example 1, the proteoglycans were detected by means of Goldner staining. Type I collagen and proteoglycans are components of the bone matrix in vivo, representing the most important structural proteins which are of crucial significance for bone function.

At the same time, proliferative bone cells were detected in the outer layer of the aggregates.

After culturing for 2 weeks, the cells are still close together. With increasing cultivation time, the proportion of extracellular matrix increases and the proportion of cells decreases. After one week, at least 40% ECM can be detected, and after 3 weeks, about 60% ECM has already developed. That is, bone-like tissue has been built up inside the aggregates produced, which tissue in its structure corresponds to in vivo bone and is also capable of assuming the function of bone tissue.

The single components thus obtained are now ready to be combined with cartilage suspension cells/single cells. The growth factors produced and secreted by the cells in the three-dimensional in vitro tissues serve in promoting the de novo regeneration of the joint cartilage or bone structure and thus in increasing the efficiency in the treatment of cartilage or bone tissues.

Combination of Preformed Three-Dimensional Tissue (Spheroids) from Bone Cells using Electromagnetic Fields

During the production of the bone cell-based spheroids and/or subsequent to incorporating the bone spheroid in affected, degenerate or destroyed tissue, the tissue or the tissue-regenerating processes are stimulated in vivo by means of electromagnetic fields. Remarkably, it has been determined that maturing of the spheroids produced from bone cells is stimulated when applying an electromagnetic field with a carrier frequency of 5 kHz and various modulation frequencies (for example 16 Hz). Furthermore, it is possible to combine the spheroids with growth factors. Surprisingly, it has been determined that growth of cartilage cells and also, matrix formation and maturing can be influenced favorably upon addition of exogenic growth factors during the production of spheroids from cartilage cells.

Preparation of Spheroids from Genetically Engineered Cartilage Cells, in Combination with Cartilage Cells in Suspension

It has been demonstrated that maturing of the cartilage tissue having formed is promoted in infections of human cartilage cells and in the production of spheroids therefrom. In clinical use, in particular, this implies more rapid healing of defects or tissues in regeneration.

Combination of Spheroids with PLA/PGA Polymers

The spheroids produced from bone cells are used in coating or growing into the support material, e.g. neutrally degrading PLA/PGA polymers and collagen fleeces implanted as structural substances in tissue engineering. It has been demonstrated that subsequent to addition of spheroids, produced from bone cells on the surface of neutrally degrading PLA/PGA polymers, said spheroids grow across the surface, forming a final layer, but also migrate into the polymers. For clinical use, more rapid healing of a defect and more rapid rearrangement of the neutrally degrading PLA/PGA polymer is achieved in this way. The same has been shown for a combination of spheroids from bone cells with collagen membrane.

Meniscus

Preformed three-dimensional meniscus cartilage tissue is produced as described for cartilage tissue and combined with a support material outside the body, e.g. during operation, which material confers mechanical stability and shape.

Muscle

Three-dimensional muscle cells are produced in analogy to the production of cartilage cells and combined with an autologous muscle cell suspension consisting of endogenous cardiac muscle cells or stem cells and further comprising endogenous serum, but without addition of growth-promoting compounds. Instead of the autologous muscle cell suspension of endogenous cardiac cells and/or stem cells, the three-dimensional preformed tissue can also be applied on a membrane, to be subsequently incorporated in or coated on the muscle defect.

Connective Tissue Cells

Another example relates to the preparation of spheroids from connective tissue cells modified by genetic engineering in a way so as to include a vector for insulin synthesis. The spheroids produced from these cells are encapsulated in an inert support material allowing diffusion of insulin therethrough and to the outside. This combination is implanted in the blood-supplying artery. Owing to the high cell concentration in the spheroids, this procedure allows particularly high insulin liberation, thereby increasing the therapeutic effect.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102 53 066.1, filed Nov. 7, 2002 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A tissue replacement structure, characterized in that the structure comprises
(a) a preformed three-dimensional tissue which can be produced by obtaining cells from a human or animal organism and culturing them in a stationary fashion as a suspension culture in cell culture vessels with hydrophobic surface and tapering bottom until a cell aggregate is formed which has differentiated cells embedded therein and has an outer region wherein cells capable of proliferation and migration are present;
(b) (i) an autologous cell suspension which can be produced from endogenous cells, with endogenous serum being added, with no addition of growth-promoting compounds, (ii) implants or support materials and/or (iii) growth factors; and/or
(c) can be obtained by exposure of the tissue according to (a) to electromagnetic fields, mechanical stimulation and/or ultrasound.

2. The tissue replacement structure according to claim 1, characterized in that the tissue replacement structure is a cartilage replacement structure, said tissue cell suspension being a cartilage cell suspension, said three-dimensional tissue being a cartilage tissue, with cartilage cells, bone cells and/or mesenchymal stem cells being obtained from said organism, and said cell aggregate containing at least 40% by volume of extracellular matrix.

3. The tissue replacement structure according to claim 1, characterized in that the structure is a replacement structure for muscle tissue, bone tissue, connective tissue, skin tissue, fat tissue, nervous tissue, liver tissue, endothelial and/or epithelial tissue, particularly a cardiac smooth muscle tissue replacement structure.

4. A tissue replacement structure selected from the group comprising muscle, connective, skin, fat, nervous, liver tissues, endothelia, epithelia, and/or stem cells, characterized in that the structure can be produced by obtaining cells from a human or animal organism and culturing them in a stationary fashion as a suspension culture in cell culture vessels with hydrophobic surface and tapering bottom until a cell aggregate is formed which has differentiated cells embedded therein and has an outer region wherein cells capable of proliferation and migration are present.

5. A method for the modification of a tissue lesion, characterized in that
(a) a preformed three-dimensional tissue which can be produced by obtaining cells from a human or animal organism and culturing them in a stationary fashion as a suspension culture in cell culture vessels with hydrophobic surface and tapering bottom until a cell aggregate is formed which has differentiated cells embedded therein and has an outer region wherein cells capable of proliferation and migration are present; and
(b) an autologous cell suspension which can be produced from endogenous cells, with addition of endogenous serum and without adding growth-promoting compounds,
are incorporated in the tissue lesion and/or
(c) exposure of the tissue according to (a) to electromagnetic fields, mechanical stimulation and/or ultrasound is effected.

6. The method according to claim 5, characterized in that the tissue lesion is a bone, cartilage and/or muscle lesion.

7. The method according to claim 6, characterized in that in said modification of a cartilage lesion, a cartilage cell suspension is produced as cell suspension, a cartilage tissue is produced as three-dimensional tissue, with cartilage cells, bone cells and/or mesenchymal stem cells being obtained from the organism, and the cell aggregate including at least 40% by volume of extracellular matrix.

8. The method according to claim 7, characterized in that incorporation of the cartilage cell suspension and cartilage tissue is followed by covering the lesion with a membrane.

9. Use of cartilage cells, muscle cells, bone cells, and/or mesenchymal stem cells, which cells are obtained from a human or animal organism and cultured in a stationary fashion as a suspension culture in cell culture vessels with hydrophobic surface and tapering bottom until a cell aggregate is formed which has differentiated cells embedded therein and has an outer region wherein cells capable of proliferation and migration are present, as a source of intracellular messenger substances, structural, scaffold and/or matrix components.

10. The use according to claim 9, characterized in that the intracellular messenger substances are growth factors and/or cytokines.

11. The use according to claim 9, which use is in vivo or in vitro.

12. Use of a tissue replacement structure according to claim 1 in the treatment of a tissue lesion.

13. The use according to claim 12, characterized in that the tissue lesion is a cartilage, bone and/or muscle lesion.

14. Use of a tissue replacement structure according to claim 1 as an in vitro or in vivo test system, particularly in screening of active substances.

15. A kit, comprising at least one tissue replacement structure according to claim 1, optionally together with information on combining the contents of the kit.
